(19) United States
(12) Patent Application Publication
(10) Pub. No.: US 2012/0022890 A1
(43) Pub. Date: Jan. 26, 2012

(54) METHOD AND APPARATUS FOR A SELF-SERVICE KIOSK SYSTEM FOR COLLECTING AND REPORTING BLOOD ALCOHOL LEVEL

(76) Inventors: Barry Williams, Orion Township, MI (US); Raymond Wiggins, Lake Orion, MI (US)

(21) Appl. No.: 12/448,266

(22) PCT Filed: Dec. 13, 2007
(86) PCT No.: PCT/US07/25509

§ 371 Date: Oct. 2, 2009

Related U.S. Application Data
(60) Provisional application No. 60/869,780, filed on Dec. 13, 2006.

(51) Int. Cl. A61B 5/097; H04N 7/18; G06Q 50/00; G01N 1/22; H04L 9/32

(52) U.S. Cl. 705/3; 422/84; 600/532; 705/2; 726/4; 348/77; 348/E07.085

(57) ABSTRACT

A method and an apparatus is provided for a self-service breathalyzer kiosk system for collecting and reporting blood alcohol level information. The system and method enables a user to submit a report containing the user's current blood alcohol level at the time the report is submitted. In particular, the kiosk may be used to collect the current blood alcohol level information of the user and transmit the report of the current blood alcohol level information of the user over a communication network.

### CROSS REFERENCE TO RELATED APPLICATION

This application claims priority to and all the benefits of U.S. Provisional Application Ser. No. 60/869,780, filed on Dec. 13, 2006 and entitled “PO 1000.”

### BACKGROUND OF THE INVENTION

1. Field of the Invention

The invention relates to a method and an apparatus for a self-service breathalyzer kiosk system for collecting and reporting blood alcohol level information. In particular, the system and method of the invention may be used to collect the current blood alcohol level information of a user and report current blood alcohol level information of the user to a probation or parole officer over a communications network through the use of the self-service breathalyzer kiosk.

2. Description of Related Art

While it is possible to determine an individual user's blood alcohol level by analyzing a sample of an individual user's breath, many testing facilities require the use of devices located on their premises, which is often inconvenient for the user. In addition, presently available self-administered breath testing devices do not make provision for the security of the results. Further, the blood level results that are acquired on the premises of testing facilities are often unavailable to other facilities that may need to access those results immediately.

It is therefore a goal of the invention to securely collect and transmit the user's blood alcohol level information in a manner that does not require the user to go on-site to a testing facility such as a police station and is thus more convenient for the user. It is further the goal of the invention to make available the result of the user's blood alcohol level testing accessible through a secure connection to the Internet and to other communication networks.

### SUMMARY OF THE INVENTION

According to one aspect of the invention, a method and an apparatus is provided for a self-service breathalyzer kiosk system for collecting and reporting blood alcohol level information. The system and method enables a user to submit a report containing the user's current blood alcohol level at the time the report is submitted. In particular, the kiosk may be used to collect the current blood alcohol level information of the user and transmit the report of the current blood alcohol level information of the user over a communication network.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 illustrates a self-service breathalyzer kiosk system with network connections in accordance with one embodiment of the invention;

FIG. 2 illustrates a front perspective view of one embodiment of a self-service breathalyzer kiosk;

FIG. 3 illustrates a simplified block diagram of the self-service breathalyzer kiosk system;

FIG. 4 illustrates a simplified block diagram of a central server system;

FIG. 5 illustrates a simplified flow diagram of a process for collecting and reporting the results of a breathalyzer test in accordance with the self-service breathalyzer kiosk.

### DETAILED DESCRIPTION OF THE PREFERRED EMBODIMENTS

A method and an apparatus for a self-service breathalyzer kiosk system for collecting and reporting blood alcohol level information are disclosed. The system and method of the present invention in the embodiment described below enables a user such as a convicted parolee (a “user”) to submit a report containing the user's current blood alcohol level at the time the report is submitted. In particular, the embodiment described may be used to collect the current blood alcohol level information of the user and report the current blood alcohol level information of the user to a probation or parole officer (an “officer”) over a communications network. This collecting and reporting of the current blood alcohol level of the user is accomplished through the use of the self service breathalyzer kiosk system.

In this embodiment, the process illustrates how to securely collect and transmit the user's blood alcohol level information in a manner that does not require the user to go on-site to a processing facility such as a police station and is thus more convenient for the user. In this and other embodiments, additional functions of the breathalyzer kiosk system may include software updates by a system administrator, registration of permitted users by authorized officers, registration of officers permitted to interact with the kiosk system by a system administrator, and the generation of and search for reports related to the usage of the kiosk system.

In one embodiment, a breathalyzer kiosk in the breathalyzer kiosk system is a client as known in the relevant art and executes software in conjunction with a central server system. It is contemplated that the breathalyzer kiosk may be a stand-alone unit that executes its software programs independent of any other computers or servers or alternatively as a part of other types of network architectures known in the art, for example, as a peer-to-peer network, that is, as a system of kiosk-to-kiosk networks. The breathalyzer kiosk in the breathalyzer kiosk system may also be coupled to other devices such as personal computers, personal digital assistants (PDAs), mobile phones, user interfaces, printers, cameras, input-output devices, biometric scanners, card readers, and monetary input devices.

The following description discusses the invention in the context of the Internet and communications networks; however, it is readily apparent to one of skill in the relevant art that the invention is not limited to these particular networks, and is applicable to any network that is configured to transmit data from one system or device to another connected system or device, for example, as through a network of wireless radio frequency connections.

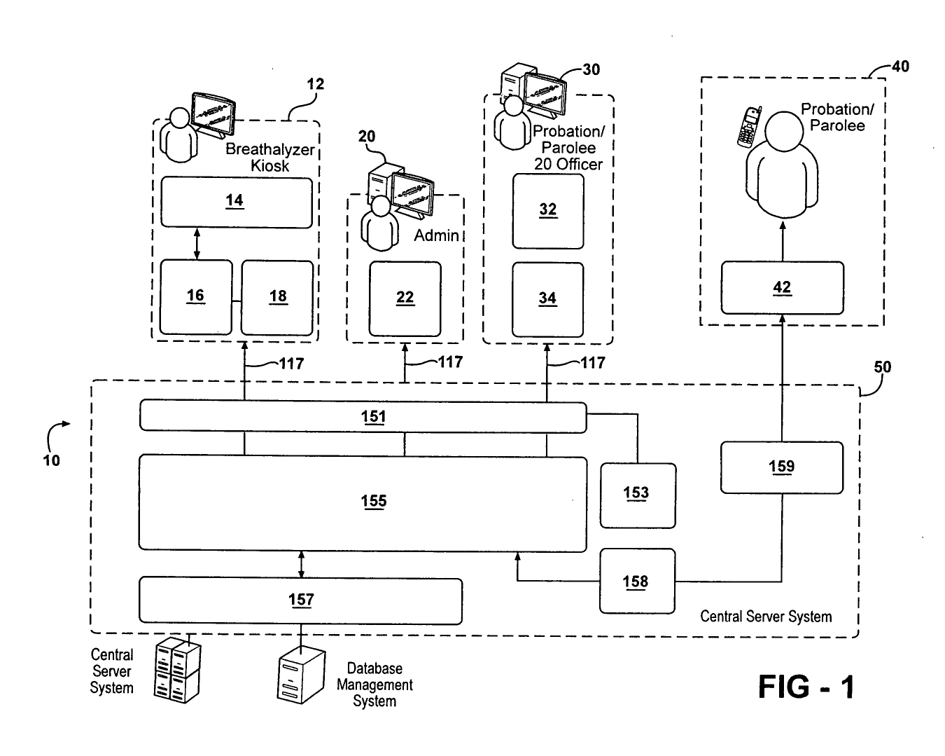

FIG. 1 illustrates a self-service breathalyzer kiosk system 10 configured in accordance with one embodiment of the invention. In one aspect of this embodiment, a breathalyzer kiosk 12 is coupled to a central server system 50. Also coupled to the central server system 50 are an administration system 20, an officer interface system 30, and a user notification system 40. In one embodiment, the administration system 20 and the officer interface system 30 each include a personal computer having a known interne browser interface and known software applications programmable to interact with and exchange information with the central server system 50.

It is also contemplated that in an embodiment of the breathalyzer kiosk system 10, either the administration system 20 or the officer interface system 30 may include in place of the personal computer a mobile device such as a personal digital assistant or a mobile phone having a known interne browser interface and known software applications programmable to interact with and exchange information with the central server system 50. It is further contemplated that in an embodiment of the breathalyzer kiosk system 10, the administration system 20 and the officer interface system 30 may both include in place of personal computers mobile devices such as personal digital assistants or mobile phones having interne browser interfaces and known software applications programmable to interact with and exchange information with the central server system 50.

While FIG. 1 illustrates a breathalyzer kiosk system 10 having a single breathalyzer kiosk 12 coupled to the central server system 50, other embodiments are contemplated and anticipated that may have a plurality of breathalyzer kiosks 12 configured according to the description provided below that are all coupled to the central server system 50 or coupled to a plurality of central server systems 50.

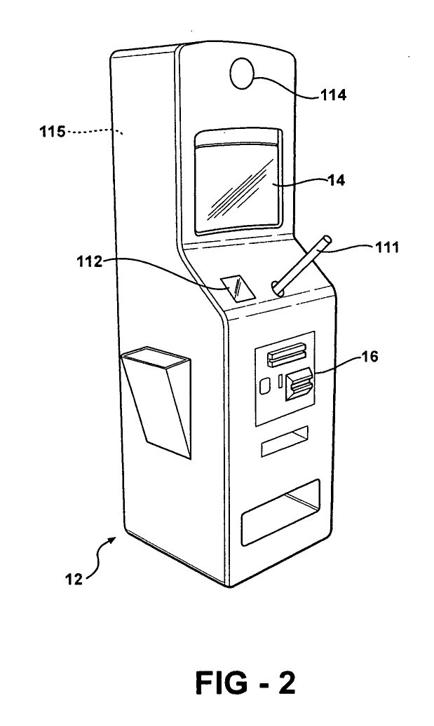

FIG. 2 illustrates a front perspective view of a breathalyzer kiosk 12 configured in accordance with one embodiment of the invention. In one aspect of this embodiment, the breathalyzer kiosk 12 includes a display 14, a hardware unit 18, a business transaction system 16, and a known power source (not shown).

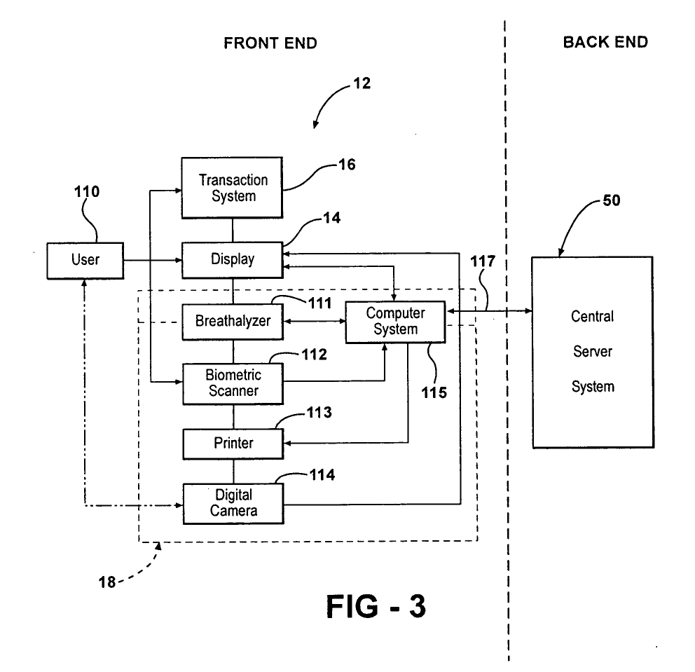

The display 14 interfaces a user 110 with the breathalyzer kiosk 12 as shown in FIG. 3. The display 14 enables the kiosk 12 to send output to a screen viewable by the user 110 such as to display the status of the alcohol blood level collection and reporting process or to display instructions on how to use the kiosk 12. The display 14 may also provide output requests for prompting the user 110 to provide input. In one embodiment, the display 14 is preferably a touch sensitive screen that enables the user 110 to provide the input by directly interacting with the display 14. For example, by touching a graphical representation of a keyboard on the screen of the display 14 illustrated in FIG. 2, the user 110 may input personal information such as name, address, telephone number, and such.

It is contemplated, however, that in other embodiments a variety of known user interfaces may be used. For example, the kiosk 12 may use a wireless communication device for input and output, an input device such as a keyboard or keypad may be coupled to the display 14, a pen-based user interface may also be used for inputting information, or a speech-recognition device may be coupled to the display 14.

The hardware unit 18 of the kiosk 12 as illustrated in FIG. 3 includes a breathalyzer 111, a biometric scanner 112, a printer 113 for printing alphanumeric data and other suitable data, a digital camera 114, and a computer system 115. The devices 111, 112, 113, 114, 115 of the hardware unit 18 interact to carry out essential functions of the kiosk 12 as is discussed in detail below. The display 14 is connected to the computer system 115 and controlled by a suitable application software program stored in the computer system 115. The application software program can be programmed by one of ordinary skill in the computer programming art to perform the desired functions of the computer system 115.

The computer system 115 preferably comprises a known embedded system having a known operating system such as Microsoft Windows CE or a Linux based embedded system package. The computer system 115 further comprises known non-volatile computer memory or other suitable low power memory technologies such as flash memory and a communications mechanism for transmitting and receiving data over a communications link 117, as shown in FIG. 3. The communications link 117 is preferably a known T1 line for transmitting voice and data signals using appropriate transmissions protocol and a telephone line as a back-up connection for dial-up-modem connectivity; however, it is contemplated that in other embodiments, the communications link 117 may be wireless.

While the embedded system and the flash memory offer reduced size, lower cost, and increased reliability, it is contemplated and anticipated that other embodiments of the kiosk 12 may include at least one personal computer as conventionally known in the relevant art having a central processing unit, at least one memory device, an operating system, an input/output interface, and a communication interface for accessing the internet and other networks. The at least one memory device is optimally configured in a redundant array of independent drives (RAID) scheme as known in the relevant art for ensuring that the kiosk system 10 continues to function when there is a failure in the at least one memory device.

The computer system 115 contains appropriate application software programs and memory for storing and processing data, for interacting with the central server system 50, for maintaining security by transmitting and receiving data using known encryption schemes such as secure socket layer (SSL) and transport layer security (TSL) protocols. All data transferred from the computer system 115 to the central server system 50 is encrypted and remains encrypted throughout transmission.

The breathalyzer 111 is a known device for accurately determining the blood alcohol level of the user 110 by receiving and analyzing a breath sample from the user 110. The breathalyzer 111 receives the breath sample and after electrochemical processing and other suitable means for testing the blood alcohol level the breath sample produces an output signal that is representative of the blood alcohol level of the user 110. The breathalyzer 111 is connected to the computer system 115 and the output signal of the breathalyzer 111 that is preferably an actual numeric value of a precise blood alcohol level of the user 110 is stored in the memory of the computer system 115, is transferred to the central server system 50 for subsequent retrieval, or both stored in the memory of the computer system 115 and simultaneously transferred to the central server system 50.

It is contemplated that alternatively the output signal of the breathalyzer 111 may also be stored on a more permanent storage device commonly known in the relevant art such as a compact disc read-only memory (CD-ROM) device. It is further contemplated that the output from the breathalyzer 111 may be printed in alphanumerical characters from the printer 113 on paper and retained for backup purposes.

The biometric scanner 112 of the hardware unit 18 is a known biometric input device for receiving biometric identifying information from the user 110. The biometric scanner 112 preferably comprises a known fingerprint scanning device connected to the computer system 115 for transferring biometric data such as a fingerprint scan received from the biometric scanner 112 to the computer system 115 or to the central server system 50 for storing in memory or for carrying out various security functions. For example, the fingerprint scanning device may be connected to the computer system 115 having a suitable software program for comparing biometric data received from the user 110 and an authorized fingerprint recognition sample that was entered into and stored on the computer system 115 or the central server system 50 during an initialization or registration phase. Therefore, to access the breathalyzer kiosk 12, the user 110 must touch the fingerprint scanning device and wait for the software program to confirm that the user 110 is authorized to access the breathalyzer kiosk system 10.

It is contemplated that alternative embodiments of the breathalyzer kiosk 12 will comprise other known biometric data input devices without departing from the scope of the present invention such as palm scanning devices, facial image devices for scanning the human face, voice recognition devices, and retina scanning devices.

The breathalyzer kiosk 12 includes the digital camera 114 for taking still digital pictures and streaming video while the user 110 is interacting with the breathalyzer kiosk 12.

The breathalyzer kiosk 12 accepts various forms of payment, including cash, credit cards, debit cards, smart cards, and other known magnetic strip cards through the business transaction system 16. The transaction system 16 interfaces with the central server system 50 for performing associated financial transactions such as confirming sufficient funds to perform blood alcohol level testing and collecting or transferring fees required to complete the testing. It is contemplated that the transaction system 16 will be compatible with a variety of financial processing systems and therefore may include provide a magnetic strip reader, a smart card chip reader, and a cash input device, as illustrated in FIG. 2.

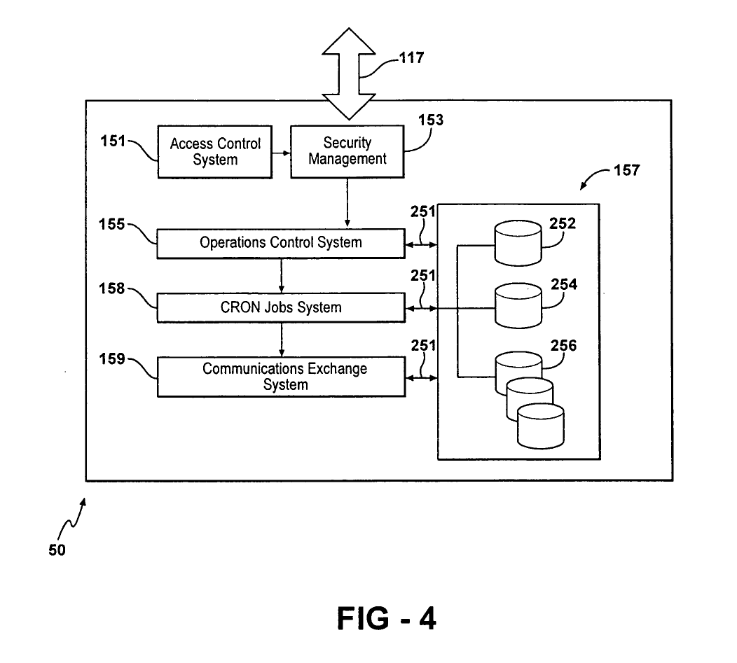

FIG. 4 illustrates an embodiment of the central server system 50. The central server system 50 is a known server having a conventionally known operating system such as a Linux, UNIX or UNIX-like system and is connected to the kiosk 12 through the communications link 117. Information provided by the user 110, data collected from the user 110, and transactions initiated by the user 110 at the breathalyzer kiosk 12 are routed to the central server system 50 through the communications link 117.

In the embodiment shown, the central server system 50 includes an access control system 151, a security management system 153, an operations control system 155, a data management system 157, a cron jobs system 158 and a communication exchange system 159. Although in the present embodiment the central server system is depicted in FIG. 4 as a single server with the functionality of the access control system 151, the security management system 153, the operations control system 155, the data management system 157, the cron jobs system 158, and the communication exchange system 159 all incorporated into the single server, a person of skill in the relevant art should recognize that the central server system 50 may include a plurality of servers with the functionality of each of the incorporated systems 151, 153, 155, 157, 158, 159 stored on, implemented by, and distributed among any of the plurality of servers.

The access control system 151 is coupled to the central server system 50 and contains appropriate application software programs for authorizing the user 110 to access the kiosk system 10 to perform breathalyzer testing. The access control system 151 facilitates user 110 security by authenticating the user 110 prior to using the kiosk system 10. A known means of authentication includes using a username and password or PIN code entry process. Authentication may also be achieved by using more sophisticated technologies such as known biometric devices used to conduct fingerprint recognition procedures as discussed below.

The access control system 151 preferably includes a biometric process for validating that the user 110 interacting with the kiosk 12 is an authorized user 110. For example, this can be achieved by using a known fingerprint recognition pad or scanner as discussed above. This process involves capturing the fingerprint scan, executing a comparison algorithm against an authorized fingerprint sample that is stored in a database on the data management system 157. If a match is found, the user 110 is authorized to access and use the kiosk 12. The comparison algorithm can be programmed by one of ordinary skill in the computer programming art to perform a comparison function.

The security management system 153 ensures secure communications among the components internal to the breathalyzer kiosk 12, the central server system 50, and between the breathalyzer kiosk 12 and the central server system 50. The security management system 153 includes participating in conventionally known secure communications protocols to open and maintain secure connections, which ensures that only authorized systems and authorized users 110 are allowed access to data from the databases contained in the central server system 50. One of ordinary skill in the relevant art will understand how network communications between clients and servers and between servers and servers are performed and the use of security processes to facilitate a secure and robust communications environment.

The security management system 153 has further functions that include internal system monitoring and system management. The security management system 153 includes the memory and appropriate application software programs to perform various monitoring and management services such as logging all accesses to and interactions with the kiosk system 10 including access by users 110, officers, and administrators. The monitoring and management services further include detecting common hardware-, software-, or network-related failures such as the printer 113 is out of ink, or the breathalyzer kiosk 12 is out of service, and detecting potentially fraudulent activities such as tampering with the kiosk 12. If any such issues are detected, the service management system 153 will generate a suitable visual or audio indication to prompt the user 110 to proceed to the nearest police station or other appropriate location to have the breathalyzer testing done manually.

The operations control system 155 is coupled to the access control system 151 and the security management system 153 on the central server system 50 and is connected to the kiosk 12, the administration system 20, the officer interface system 30, and the user notification system 40 through the communications link 117. The operations control system 155 further includes appropriate application software programs for performing the desired control functions of the breathalyzer kiosk system 10.

The data management system 157 includes at least one database stored on the central server 50 and appropriate database management software commonly known in the relevant art for managing the database such as MySQL, Postgresql, etc. It is anticipated and contemplated that the data management system 157 may not reside on the central server system 50, but may instead reside on a separate server (not shown) without departing from the scope of this invention. In addition, the computer system 115 as shown in FIG. 3 stores and executes software that one of ordinary skill in the computer science art would understand to be capable of retrieving data collected in the at least one database stored on the central server 50 or on databases stored on separate servers.

Referring to FIG. 4, the data management system 157 includes a user database 252 for collecting relevant information about all registered users such as personal information about the user 110, an assigned username and password, or PIN number for authorization purposes, a fingerprint scan, and such. As discussed in detail below, this information is provided when the user 110 is registered by a probation officer to be scheduled for periodic alcohol blood level testing or by an authorized administrator. The data management system 157 further includes a kiosk database 254 for collecting information related to each kiosk 12 coupled to the central server system 50 including information such as a unique identifier assigned to each kiosk 12 for security purposes. The data management system 157 further includes at least one department database 256 for collecting, organizing, and accessing relevant information such as administrative information, officer information, and user information by office and by departments within that office.

Data stored in the data management system 157 as described above may include text-based documents, images, hypertext markup language (HTML) documents, extensible markup language (XML) documents and the suitable software to enable rendering these documents appropriately. It is well known by one skilled in the relevant art how the data management system is searchable using various classifications of the data stored in the data management system 157.

The administration system 20 illustrated in FIG. 1 includes at least one personal computer 22 having a known interne browser or a suitable software program for connecting to the central server system 50 and/or to the kiosk 12 through the communications link 117. Information and data exchanged between the administration system 20 and the central server system 50 are fully encrypted during transmission. As discussed above, it is contemplated that the at least one personal computer 22 may be substituted with a conventionally known mobile device for connecting to the central server system 50 or to the kiosk 12.

The at least one personal computer 22 of the administration system further includes a suitable software program programmable by one of ordinary skill in the computer programming art for interacting with the operations control system 155 of the central server system 50 to perform such administrative service functions as described below. The administrative service functions include designating an administrator in an office or department, adding a new officer to the kiosk system 10, editing account information pertaining to a new officer, an existing officer, a new user, or an existing user, resetting passwords, assigning or reassigning the officer to a user for restricting that officer's access to information pertaining to that user only, monitoring the status of the kiosk 12, remotely interacting with the kiosk 12 for troubleshooting, maintenance, and updating software, monitoring the status and capacity of the central server system 50, and performing backups and updates for the data management system 157.

The officer interface system 30 includes a known biometric scanner 32 and at least one personal computer 34 having a known interne browser or a suitable software program for connecting the central server system 50 and/or to the kiosk 12 through the communications link 117. The biometric scanner 32 is coupled to the at least one personal computer 34. Information and data exchanged between the officer interface system 30 and the central server system 50 are fully encrypted during transmission. And for additional security, the officer interface system 30 may be assigned a static IP address for connecting to the central server system 50 or kiosk 12. The static IP address would only permit the officer interface system 30 to access records of users 110 associated with a particular office or department. As discussed above, it is contemplated that the at least one personal computer 34 may be substituted with a mobile device for connecting to the central server system 50 or to the kiosk 12.

A use scenario of the officer interface system 30 within the kiosk system 10 includes the registration of new users 110 permitted to access and use the kiosk system 10. For example, an officer completes a form preferably an internet-based form on the at least one personal computer 34 providing the relevant information about the user 110. Fingerprint samples of the user 110 are scanned into the biometric scanner 32. Digital photographs are taken by the officer. As discussed above, it is contemplated that alternate embodiments may include other biometric devices for receiving biometric input without departing from the scope of the invention. The officer further inputs a breathalyzer testing schedule for the user 110. The completed form, the fingerprint sample, the photographs, and the breathalyzer testing schedule along with associated security information are transmitted to the central server system 50 to be stored in the appropriate databases within the data management system 157.

The officer interface system 30 is further coupled to the cron jobs system 158 having a suitable software program programmable by one ordinarily skilled in the programming art to execute scheduled automated events. The breathalyzer testing schedule transmitted by the officer when registering new users may be used by the cron jobs system 158 to generate automated email notifications to officers concerning test results of users 110 and make automated phone calls to users 110 for requesting the user 110 to be tested.

The user notification system 40 is connected to the communication exchange system 159 on the central server system 50 and includes a known communication device 42 such as a personal computer, a personal digital assistant, or mobile phone having suitable software adapted for receiving audio files and data files such as email messages and voice files transmitted by the communication device 42. The communication exchange system 159 preferably includes a known email server and a known private branch exchange (PBX) for transmitting audio signals preferably transmitted using voice over interne protocol (VoIP) to registered users 110. The VoIP transmission facilitates security measures by allowing encryption and authentication of voice transmissions.

In one use scenario of the user notification system 40, the test schedule as discussed above set up by the officer through the officer interface system 30 initiates an automated call to the registered user 110 notifying the user 110 of a pending breathalyzer test date and time, and further notifying the user 110 of the location of the kiosk 12 or kiosks 12 that the user 110 is authorized to use.

The test schedule also initiates an automated email message to be sent to the officer on a regularly scheduled basis to notify the officer of test results for review. The notification is preferably in the form of a count of how many new results are available for review since the officer last reviewed results and the number of users 110, if any, that failed to appear for scheduled testing. The officer should be further directed to log into the appropriate application software program through the officer interface system 30 to obtain reports about the officer's assigned users 110.

Examples of the reports that officers are capable of generating include a list of all users, a list of users tested within the last twenty four hours, a list of users who were supposed to be tested, but did not complete the process in the required timeframe, and user blood alcohol level results. The reports may be formatted to include user information, photographs, current blood alcohol levels and are capable of being printed.

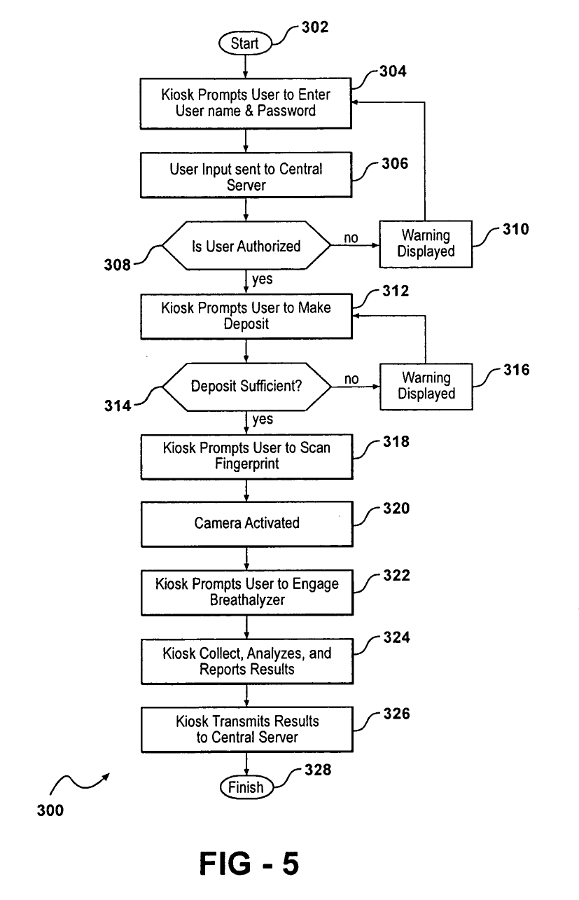

FIG. 5 is a flow diagram illustrating a process 300 by which the user 110 interacts with the breathalyzer kiosk system 10 using the kiosk 12 illustrated in FIG. 2. The process starts at block 302. At block 304, the display 14 on the kiosk 12 prompts the user 110 to enter a username and password. At block 306, the user 110 enters the username and the password and the username and password are sent to the central server system 50 for validation. At block 308, the validation process is initiated. If the user 110 is not authorized to use the kiosk 12, the display 14 of the kiosk 12 will display a warning at block 310. The user 110 is then returned to block 304 and the display 14 prompts the user 110 to enter username and password again. If validation fails continually after a finite number of tries, the kiosk 12 will be inaccessible to that user 110 and the display 14 will provide an on-screen message to the user 110 to proceed to the nearest police station or other appropriate location to have the breathalyzer testing done manually.

If the user 110 is authorized to use the kiosk 12, the display 14 on the kiosk 12 prompts the user 110 to deposit the appropriate amount of money into the kiosk 12 for conducting the breathalyzer test at block 312. At block 314, if the user 110 does not deposit a sufficient amount of money, the display 14 of the kiosk 12 will display a warning at block 316. The user 110 is then returned to block 312 and the display 14 prompts the user 110 to make a deposit again. If the user 110 fails to deposit the appropriate amount of money, after a threshold period of time, the kiosk 12 will be inaccessible to that user 110 and the display 14 will provide an on-screen message to the user 110 to proceed to the nearest police station or other appropriate location to have the breathalyzer testing done manually. Any money deposited in the kiosk 12 will be returned or any credit card or debit card transaction initiated will be cancelled.

If the deposit is sufficient, at block 318 the display 14 will prompt the user 110 to submit a finger print scan. In one embodiment in which fingerprint data is used, the user 110 is prompted to scan a fingerprint by successively pressing one or more fingers against the biometric scanner 112. The display 14 may prompt the user 110 for each fingerprint that must be scanned, for example, using a graphical image of a hand with the indicated finger, or audibly instructing the user 110 to use a specified finger on a specified hand. Once the finger print is scanned, at block 320 the digital camera 114 is activated to take a digital photograph and video stream of the user 110 taking the test. At block 322, the user 110 is prompted to engage the breathalyzer 111 with instructions on how to use the breathalyzer 111. At block 324, the user's blood alcohol level is collected and analyzed and the result is generated. At block 326, the results are transmitted to the central server system 50 for further processing as discussed above. The process ends at block 328.

One embodiment of the apparatus may be constructed using an object-oriented software application development model using C++ programming language or another suitable object-oriented programming language for implementing hardware interaction. Hardware components may utilize both off-the-shelf components as well as proprietary device technology. The integration of components may utilize a proprietary construction design intended to allow for subsequent open platform activities (such as customization of core feature sets by vendors).

The invention has been described in an illustrative manner, and it is to be understood that the terminology, which has been used, is intended to be in the nature of words of description rather than of limitation. Many modifications and variations of the present invention are possible in light of the above teachings. It is, therefore, to be understood that within the scope of the appended claims, the invention may be practiced other than as specifically described.

What is claimed:

1. An apparatus for collecting and reporting a user's blood alcohol level, said apparatus comprising:
a kiosk having a breathalyzer for receiving and testing a breath sample from the user to determine the user's blood alcohol level of the breath sample and producing an output signal representative of the blood alcohol level of the user; and a computer system coupled to said breathalyzer for storing and transmitting said output signal.

2. The apparatus of claim 1, further comprising a display having a screen viewable by the user for displaying information to the user.

3. The apparatus of claim 2, further comprising a business transaction system for accepting payment and performing financial transactions.

4. The apparatus of claim 3, further comprising a biometric scanner coupled to said computer system for processing biometric identifying information from the user.

5. The apparatus of claim 4, wherein said biometric scanner is a fingerprint scanner.

6. The apparatus of claim 5, further comprising a digital camera for capturing still digital pictures and streaming video of the user interacting with said kiosk.

7. The apparatus of claim 6, wherein said kiosk is coupled to a central server system through a communications link to exchange information and sharing resources between said kiosk and said central server system.

8. The apparatus of claim 7, wherein said kiosk is coupled to a central server system through a communications link.

9. The apparatus of claim 8, wherein said central server system comprises a data management system having at least one database.

10. The apparatus of claim 9, wherein said central server system further comprises an access control system for authorizing users to access said kiosk.

11. The apparatus of claim 10, wherein said access control system of said central server system comprises at least one biometric device for authorizing the user to access said kiosk.

12. The apparatus of claim 11, wherein said central server system further comprises a security management system for ensuring secure communications among hardware components internal to said kiosk and said central server system, and between said kiosk and said central server system.

13. The apparatus of claim 12, wherein said central server system further comprises an operations control system for controlling said kiosk.

14. The apparatus of claim 13, wherein said data management system further comprises a user database for collecting relevant information about the user.

15. The apparatus of claim 14 further comprising a plurality of kiosks coupled to said central server system, wherein said data management system further comprises a kiosk database for collecting information from each of said plurality of kiosks.

16. The apparatus of claim 15, wherein said data management system further comprises at least one department database for collecting, organizing, and accessing from at least one department.

17. The apparatus of claim 16, wherein said central server system further comprises a cron jobs system for executing scheduled automated events.

18. The apparatus of claim 17, wherein said central server system further comprises a communications exchange system coupled to said cron jobs system for executing scheduled automated audio and email transmissions.

19. The apparatus of claim 18, wherein said central server system is coupled to an administration system for performing administrative services on said central server system and said plurality of kiosks.

20. The apparatus of claim 19, wherein said administration system comprises at least one communication device for exchanging data and information and interacting with said central server system and said plurality of kiosks through a plurality of communication links.

21. The apparatus of claim 20, wherein said central server system is coupled to an officer interface system for performing administrative tasks on said central server system and said plurality of kiosks.

22. The apparatus of claim 21, wherein said officer interface system comprises a biometric scanner coupled to at least one personal computer for storing and transmitting biometric information collected from the user and for authorizing users to use said kiosk.

23. The apparatus of claim 22, wherein said central server system is coupled to a notification system, said notification system coupled to a communication exchange system and at least one communication device for generating automated notifications of breathalyzer test results

24. The apparatus of claim 23, wherein said at least one communication device provides notification to the user.

25. A method of collecting and reporting a user's blood alcohol level using a kiosk and central server system, said method comprising the steps of:
displaying instructions for using the kiosk;
accessing the kiosk;
collecting biometric information from the user for authorizing the user to use the kiosk;
testing a breath sample from the user to determine blood alcohol level and producing a result;
storing the result for subsequent retrieval and transmission;
transmitting results to the central server system.

26. The method of collecting and reporting the user's blood alcohol level as set forth in claim 25, wherein the step of accessing the kiosk includes the step of authorizing the user to access the kiosk;

27. The method of collecting and reporting the user's blood alcohol level as set forth in claim 26, wherein the step of accessing the kiosk further includes the step of depositing an amount of money into the kiosk;

28. The method of collecting and reporting the user's blood alcohol level as set forth in claim 27, further comprising the step of capturing a digital still photograph and streaming video of the user interacting with the kiosk;

29. The method of collecting and reporting the user's blood alcohol level as set forth in claim 25, further comprising the step of a generating a report from the result stored on the kiosk;

30. The method of collecting and reporting the user's blood alcohol level as set forth in claim 29, further comprising the step of storing the result on the central server system and generating a report from the result stored on the central server system.
